(19) United States
(12) Patent Application Publication
(10) Pub. No.: US 2006/0100524 A1
(43) Pub. Date: May 11, 2006

(54) Analysis apparatus and method

(76) Inventors: Gerhardus Wilhelmus Lucassen, Eindhoven (NL); Gerwin Jan Puppels, Rotterdam (NL); Marjolein Van Der Voort, Eindhoven (NL)

Correspondence Address: PHILIPS INTELLECTUAL PROPERTY & STANDARDS, 595 MINER ROAD, CLEVELAND, OH, 44143, US

(21) Appl. No.: 10/541,176

(22) PCT Filed: Dec. 4, 2003
(86) PCT No.: PCT/IB03/05732

§ 371 Date: Jun. 30, 2005

(30) Foreign Application Priority Data
Dec. 30, 2002 (EP) 02080575.0

(51) Int. Cl. A61B 6/00

(52) U.S. Cl. 600/476

(57) ABSTRACT

The present invention relates to an analysis apparatus, in particular a spectroscopic analysis apparatus, for analysing an object, such as the blood of a patient, and a corresponding analysis method. An excitation system (exs) emits an excitation beam (exb) to excite a target region and a beam separation unit (hm) separates at least part of elastically scattered radiation from inelastically scattered radiation, said scattered radiation being generated by the excitation beam (exb) at the target region. A monitoring system (lso) generates an image of the target region using the elastically scattered or the inelastically scattered radiation and defines a region of interest in said image. To increase efficiency of the recording of Raman spectra, a control unit (ctrl) is provided for controlling the excitation system (exs) such that the defined region of interest of the target region is excited and/or for controlling the detection system (dsy) such that only signals from the defined region of interest are detected, and a detection system (dsy) is provided for detecting scattered radiation from the defined region of interest generated by the excitation beam. Preferably the signal from the defined region of interest is averaged by distributing the laser excitation power over the defined region of interest area.

The present invention relates to an analysis apparatus, in particular a spectroscopic analysis apparatus, for analysing an object, such as the blood of a patient, and a corresponding analysis method.

In general, analysis apparatuses, such as spectroscopic analysis apparatuses, are used to investigate the composition of an object to be examined. In particular, analysis apparatuses employ an analysis, such as a spectroscopic decomposition, based on interaction of the matter of the object with incident electromagnetic radiation, such as visible light, infrared or ultraviolet radiation.

A spectroscopic analysis apparatus comprising an excitation system and a monitoring system is known from WO 02/057759 A2 which is incorporated herein by reference. The excitation system emits an excitation beam to excite a target region during an excitation period. The monitoring system emits a monitoring beam to image the target region during a monitoring period. The excitation period and the monitoring period substantially overlap. Hence the target region is imaged together with the excitation, and an image is formed displaying both the target region and the excitation area. On the basis of this image, the excitation beam can be very accurately aimed at the target region.

WO 96/29571 A1 discloses a system and method for optically aligning a capillary tube and an excitation laser beam for fluorescence detection applications by utilizing the Raman scatter signals of the capillary tube's contents. For example, Raman scatter by an electrophoretic separation matrix may be used for alignment in a capillary electrophoresis system. Fluorescent material may be present and may also be used for alignment purposes, but is not necessary. The invention employs a parabolic reflector, having apertures through which the capillary tube and the laser beam are guided so that they intersect, preferably at right angles and at the focal point of the reflector. The Raman scatter signals of the material within the capillary tube are collected via a series of filters and this information is used to reposition, if necessary, a focusing lens that directs the excitation beam into the reflector and the capillary tube, so that the Raman scatter signals are maximized. Maximal Raman scatter signals indicate proper alignment of the capillary tube and the excitation beam. Other signals, such as fluorescence emission from the sample, may then be gathered. Adjustment of the focusing lens may be automated so that alignment of the capillary tube and the beam is maintained throughout analysis of the tube's contents. Sequential alignment of an array of capillary tubes with an excitation beam is also disclosed.

The analysis method known from WO 02/057759 A2 for simultaneous imaging and spectral analysis of a local composition is done by separate lasers for confocal video imaging and Raman excitation. In case of application to non-invasive blood analysis the laser is aimed a particular blood vessel. The disadvantage is the use of two separate lasers for the separate confocal video microscope and the Raman system. Further, image processing software means are required for tracking blood vessels. There are also embodiments disclosed for combined imaging and Raman analysis by use of a single laser. However, the problem of finding a blood vessel in the image and recording Raman spectra of the blood vessel with high signal-to-noise ratio within a substantial overlap of time of imaging and Raman spectral analysis has not been solved yet.

It is therefore an object of the present invention to provide an optimised analysis apparatus and a corresponding analysis method for imaging and spectroscopic analysis of an object which supply an analysis and Raman spectra having a high signal to noise ratio and which allow the use of a single laser for both imaging and Raman excitation.

This object is achieved according to the present invention by an analysis apparatus as claimed in claim 1 comprising:
an excitation system for emitting an excitation beam to excite a target region,
a beam separation unit for separating at least part of elastically scattered radiation from inelastically scattered radiation, said scattered radiation being generated by the excitation beam at the target region,
a monitoring system for generating an image of the target region using the elastically scattered or the inelastically scattered radiation and for defining a region of interest in said image,
a control unit for controlling the excitation system such that the defined region of interest of the target region is excited and/or for controlling the detection system (dsy) such that only signals from the defined region of interest are detected, and
a detection system for detecting scattered radiation from the defined region of interest generated by the excitation beam.

The object is further solved by a corresponding analysis method as claimed in claim 10.

The present invention is based on the idea to use the excitation system to make the image of the target region. Elastically or inelastically scattered light generated at the target region in response to the excitation beam is used to provide the image, e.g. of a patient's skin with blood vessels. Based on the image information it can be zoomed in on the region of interest to a particular blood vessel, and Raman spectra from each pixel in the region of interest can be recorded. The idea is that the region of interest fully or almost fully covers a part of a blood vessel.

It is also possible to detect a Raman spectrum of each pixel in the whole image of the target region. On the basis of the spectral information the best region of interest with blood vessels is then selected and the zoom on the region of interest for faster acquisition time of the Raman signal is then performed.

The present invention has the advantage that a single laser for both imaging and Raman spectrum detection can be used, i.e. the Raman excitation beam is both used for exciting the target region and for imaging. Further, a large integrated Raman signal of blood in comparison with a fixed point recording can be obtained. Still further Raman spectral information can be used for target-tracking blood vessels using separate image processing means.

Preferred embodiments of the invention are defined in the dependent claims. Different embodiments of the monitoring system are defined in claims 2 and 3. In order to distinguish between different image portions, for instance to discriminate between pixels with blood and skin in the image either contrast information in the image or spectral information in the detected scattered radiation can be used.

When using contrast information in an image analysis Raman signals need not to be analysed. Blood vessels can be identified in the image by intensity contrast or intensity fluctuations contrast. The advantage is that image frame rates are usually much higher than Raman signal acquisition times which means that image analysis is faster than spectral analysis at the cost of a required image processing. When using a spectral analysis blood or skin can be identified since they have characteristically different spectral features. The advantage is a precise local molecular identification; however, a spectral analysis is slower compared to an image analysis.

The discrimination between blood and skin can be performed by monitoring the ratio of signal contribution of water to that of protein in the ROI. The water/protein ratio (WPR) is in blood considerably higher than in skin surrounding the blood vessels due to the presence of considerable content of collagen. To determine a water to protein ratio a filter can be used to separate high frequency spectral portions in a Raman signal, in particular portions comprising contributions from protein and water, from low frequency spectral portions, in particular a fingerprint spectral region.

The analysis apparatus according to the present invention can be a two-laser or a one-laser apparatus. In the two-laser apparatus one laser is used to produce the excitation beam while a different laser is used to emit the monitoring beam. For instance, the excitation beam can be a static beam for analysis on a single spot or a scanning beam, while the monitoring beam is preferably a scanning beam to form the image. In the one-laser embodiment the original output beam generated by a radiation source, i.e. a laser, is preferably split into the monitoring beam and the excitation beam by appropriate optical separation means.

According to other preferred embodiments the monitoring system can either include an confocal video microscope, in which the detection system has a confocal relationship with a confocal video microscope. Alternatively, the monitoring system can include an orthogonal polarized spectral imaging arrangement. Details of such monitoring systems are disclosed in the above mentioned WO 02/057759 A1.

It is further advantageous to average signal from the defined region of interest by distributing the laser power of the excitation laser over the defined region of interest which is also important with respect to limitations to maximum incident power.

Different embodiments of control systems include an embodiment where it is adapted for controlling said excitation system to distribute the laser power over the defined region of interest, but not over the whole (original) region of interest. Another embodiment of the control system is adapted for controlling said detection system to block unwanted signals (e.g. surrounding skin) from parts of the defined region of interest and to detect only wanted signals (e.g. blood) from the defined region of interest.

The invention will now be explained in more detail with reference to the drawings in which

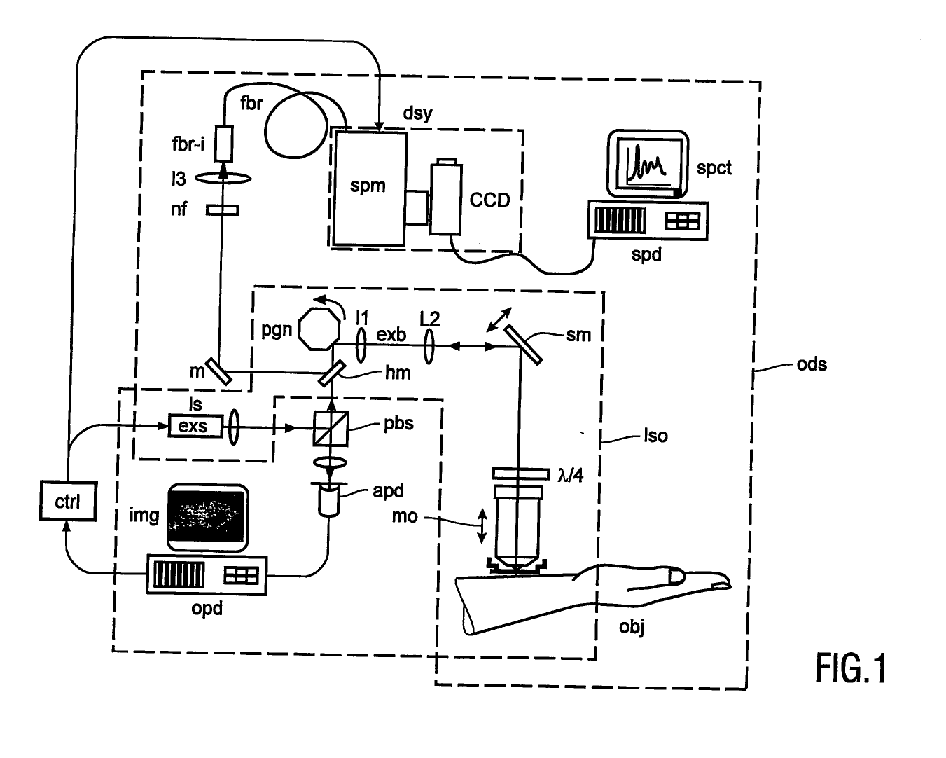

FIG. 1 shows a graphic representation of a first embodiment of an analysis system according to the present invention,

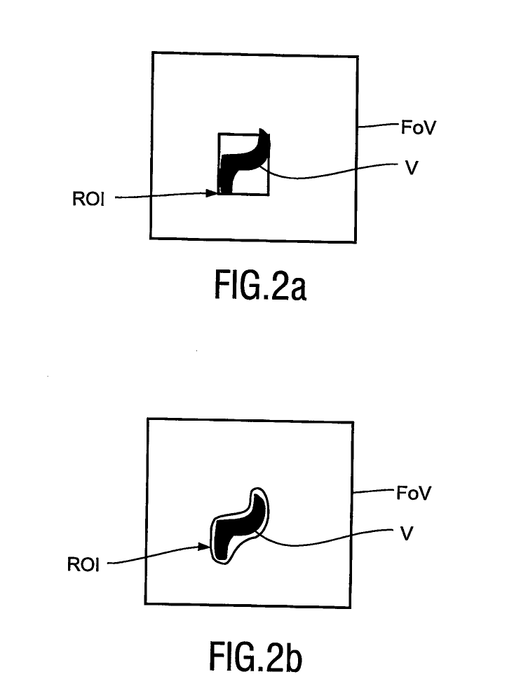

FIG. 2 illustrates the field of view and different regions of interest in the image,

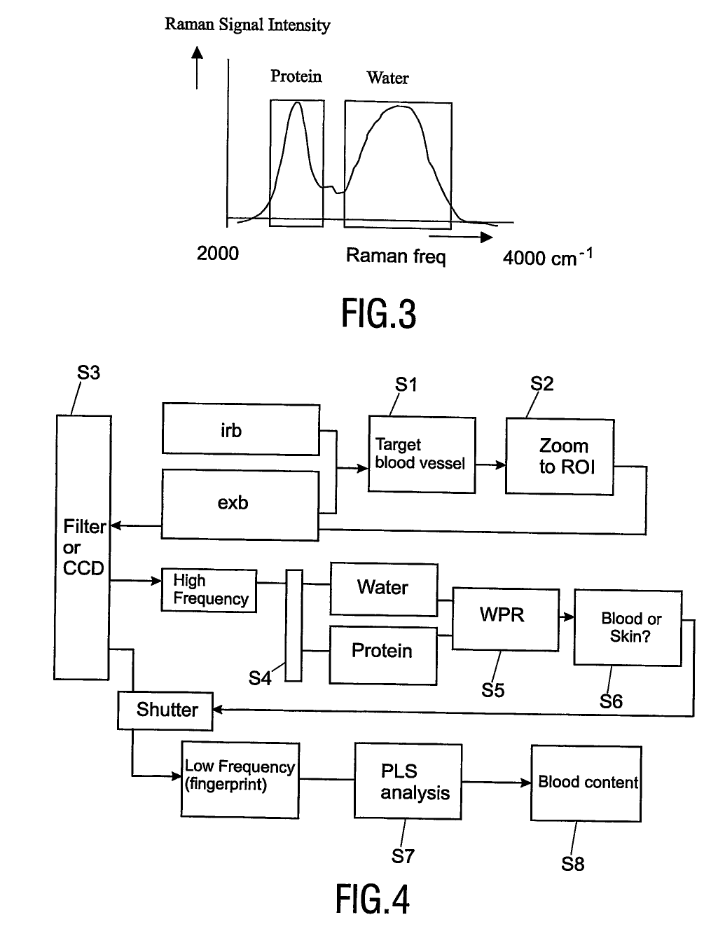

FIG. 3 shows the Raman signal intensity of high frequency components of tissue,

FIG. 4 shows a block diagram of the method according to the present invention,

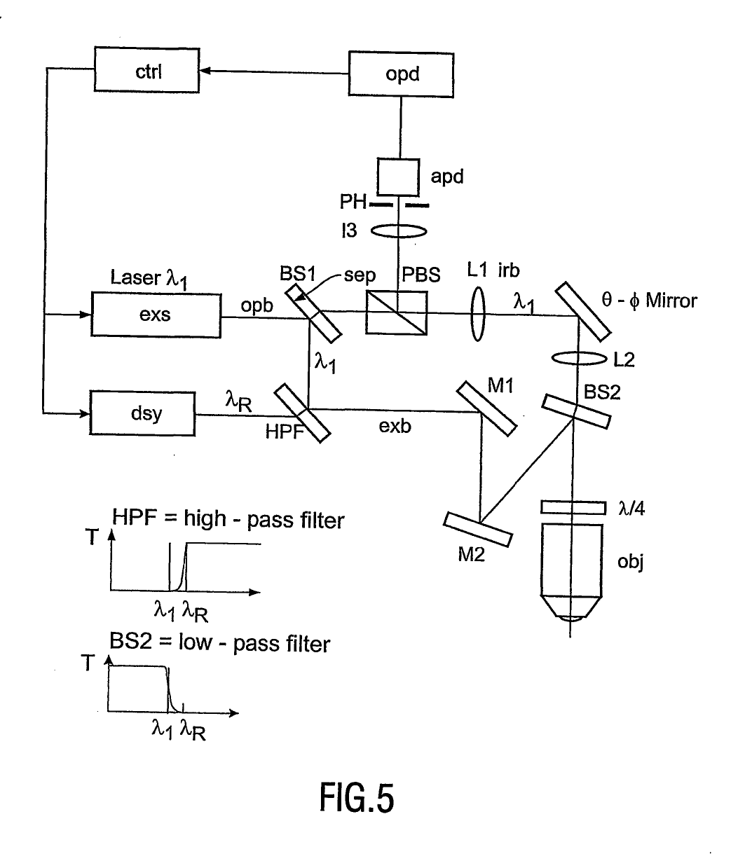

FIG. 5 shows a graphic representation of second embodiment of an analysis system according to the present invention, and

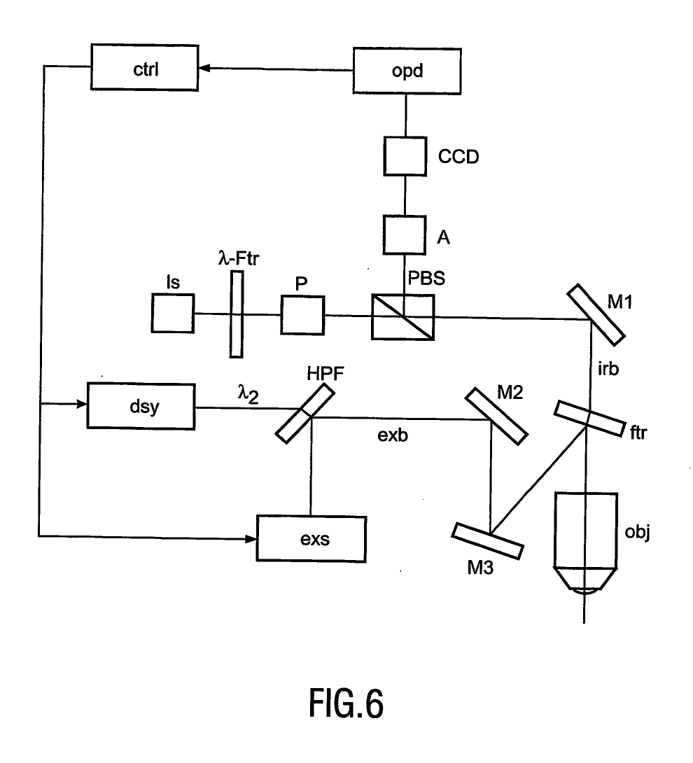

FIG. 6 shows a graphic representation of third embodiment of an analysis system according to the present invention.

FIG. 1 is a graphic representation of an analysis system in accordance with the invention. The analysis system includes an optical imaging system (lso) for forming an optical image of the object (obj) to be examined. The optical imaging system (lso) forms a confocal video microscope. In the present example the object is a piece of skin of the forearm of the patient to be examined. The analysis system also includes a multi-photon, non-linear or elastic or inelastic scattering optical detection system (ods) for spectroscopic analysis of light generated in the object (obj) by a multi-photon or non-linear optical process. The example shown in FIG. 1 utilises in particular an inelastic Raman scattering detection system (dsy) in the form of a Raman spectroscopy device. The term optical encompasses not only visible light, but also ultraviolet radiation and infrared, especially near-infrared radiation.

The light source (ls) is, for instance, formed by an Ar-ion/Ti-sapphire laser which produces the excitation beam in the form of an 850 nm infrared beam (exb). The Ti-sapphire laser is, for instance, optically pumped with the Ar-ion laser. The infrared excitation beam (exb) of the laser is focussed in the focal plane in or on the object (obj) by the optical imaging system in the exit focus. The optical imaging system includes a polarising beam splitter (pbs), a rotating reflecting polygon (pgn), lenses (11, 12), a scanning mirror (sm) and a microscope objective (mo). The focussed excitation beam (exb) is moved across the focal plane by rotating the polygon (pgn) and shifting the scanning mirror (sm). The exit facet of the semiconductor laser (ls) lies in the entrance focus. The semiconductor laser (ls) is also capable of illuminating an entrance pinhole in the entrance focus. The optical imaging system conducts the light that is reflected from the focal plane as a return beam, via the polarising beam splitter (pbs), to an avalanche photodiode (apd). Furthermore, the microscope objective (mo) is preceded by a λ/4-plate so that the polarisation of the return beam is perpendicular to the polarisation of the excitation beam. The polarising beam splitter (pbs) thus separates the return beam from the excitation beam.

An optical display unit (opd) utilises the output signal of the avalanche photodiode (apd) to form the image (img) of the focal plane in or on the object to be examined, said image being displayed on a monitor. In practice the optical display unit is a workstation and the image is realised by deriving an electronic video signal from the output signal of the avalanche photodiode (apd) by means of the processor of the workstation. This image is used to monitor the spectroscopic examination, notably to excite the target region such that the excitation area falls onto the target region and receiving scattered radiation from the target region.

The Raman spectroscopy device (ods) includes as excitation system (exs) the same laser (ls) that is used in the imaging system (lso). The Raman scatter is reflected back along the same light path as the excitation beam by the scanning mirror (sm), the lenses (11, 12) and the rotating polygon (pgn). Behind the polygon, seen in the direction of the reflected scattered light, a hot mirror (hm) is located in the light path to separate the Raman scattered light, i.e. inelastically scattered light having wavelengths different from the wavelengths of the excitation beam, from the elastically scattered light in the reflected light beam.

The Raman scattered light is directed to the entrance of a fibre (fbr) by another mirror (m), and is further focussed on the fibre entrance in the detection pinhole by a notch filter (nf) and a lens (13) in front of the fibre entrance (fbr-i). The fibre entrance itself acts as a detection pinhole. The optical imaging system (lso) establishes the confocal relationship between the entrance focus, where the semiconductor laser (ls) is present, the exit focus at the area of the detail of the object (obj) to be examined and the detection focus in the fibre entrance (fbr-i). The fibre (fbr) is connected to the input of a spectrometer (spm) with a CCD detector (CCD). The spectrometer with the CCD detector is incorporated into the detector system (dsy) which records the Raman spectrum for wavelengths that are smaller than approximately 1050 nm. The output signal of the spectrometer with the CCD detector represents the Raman spectrum of the Raman scattered infrared light. In practice this Raman spectrum occurs in the wavelength range beyond 860 nm, depending on the excitation wavelength. The signal output of the CCD detector is connected to a spectrum display unit (spd), for example a workstation which displays the recorded Raman spectrum (spct) on a monitor.

In practice the functions of the optical display unit and the spectrum display unit can be carried out by means of the same workstation. For example, separate parts (windows) of the display screen of the monitor are used for simultaneous display of the optical image and the Raman spectrum. Regarding further details of the analysis apparatus in general and the function thereof reference is made to the above mentioned WO 02/057759 A1.

According to the invention a control unit (ctrl) is provided which controls the excitation system (exs) such that only a particular defined region of interest of the target region of the object (obj) is excited and/or control the detection system (dsy) such that unwanted signals (e.g. surrounding skin) from parts of the defined region of interest are blocked and to only wanted signals (e.g. blood) from the defined region of interest are detected.

The defined region is thereby generated by the monitoring system (opd) by use of contrast information or by use of spectral information in the detected scattered radiation received from the detection system (ods). Thus, according to the present invention a full field of view (FOV), as shown in FIGS. 2a and 2b, is imaged by use of, in this particular embodiment, elastically scattered light of the excitation beam Thereafter, in the image a region of interest (ROI) having a smaller field of view is defined, the region of interest (ROI) including for instance a blood vessel V as shown in FIGS. 2a and 2b. Thereby, the region of interest can be adopted to the size and shape of the object (V) as shown in FIG. 2b or can be a rectangle as shown in FIG. 2a.

Thereafter, the scanning of the excitation beam is set to the limited size region of interest (ROI) by use of the control unit (ctrl) and only scattered radiation from this region of interest is collected. In this particular embodiment, only inelastically scattered radiation is detected by the Raman detection system (dsy). Thus, for all pixels in the region of interest (ROI) the Raman signal is collected from blood resulting in a larger Raman signal compared to known analysis methods.

When zoomed in on the ROI to cover almost fully a blood vessel or parts of a bloodvessel as shown in FIG. 2a it is the intention to discriminate between blood and skin and to only detect signal from blood area. That is, in pixels in the ROI that are not blood, either the excitation or detection is blocked. The discrimination between blood and skin can be performed by monitoring the ratio of signal contribution of water to that of protein in the ROI. The water/protein ratio (WPR) is in blood considerably higher than in skin surrounding the blood vessels due to the presence of considerable content of collagen.

Usually the characterization of tissue or blood is determined from the fingerprint spectral region (0-2000 cm⁻¹). The high-frequency spectral region 2000-4000 cm⁻¹ contains both bands of protein and water. The Raman intensity in these bands can be easily determined to perform the monitoring in each pixel in the ROI.

A filter that splits low and high frequency spectral regions can be used to generate the fingerprint and water/protein spectral regions. The WPR can be determined by integrating signals in the protein band and in the water band to deliver the two signals. This can be implemented by using filters splitting the high-frequency spectral portions as shown in FIG. 3 from low-frequency spectral portions or by reading out the corresponding pixels from the CCD camera.

A block diagram showing the main steps of an embodiment of the analysis method according to the invention is shown in FIG. 4. When using image analysis the finding of blood vessels in skin is performed by selection of pixel intensity contrast, e.g. in orthogonal polzarized spectral imaging (OPSI) or pixel intensity fluctuation in confocal scanning laser microscopy (CSLM). When using spectral analysis the blood vessel is found by selection of spectral characteristics of blood. Either method or combinations can be used to locate and select the best target blood vessel (step S1) for Raman measurements.

After selection of a blood vessel the zoom is performed (S2) to select a smaller FOV with (part) of the blood vessel as shown in FIGS. 2a,b. This can be done by different methods:

Using 2 lasers: the image scanning beam (monitoring beam, irb) is zoomed to the defined ROI, and the fixed static Raman beam (excitation beam, exb) is zoomed on a fixed point in the blood vessel (S2).

Using 2 lasers: both image scanning beam (irb) and Raman excitation scanning beam (exb) are zoomed to the defined ROI area. Raman signal is collected and averaged over all pixels in the ROI, since Raman excitation laser power is distributed over the whole ROI area instead of only directed to a fixed point. A filter is used for low frequency region and high frequency region (S3). From the high frequency region a WPR is determined and monitored (S5) using filtering (S4). Therefrom skin or blood pixels can be detected (S6). When using WPR monitoring to detect whether a skin or blood pixel is targeted the skin to blood ratio can be improved by only collecting Raman signals from blood pixels and blocking excitation or detection for skin pixels.

Using 1 laser: the Raman excitation beam (exb) is zoomed to the defined ROI. Part of the excitation beam to generate elastic light scatter for image analysis of the defined ROI and to detect skin and blood; another part is used for inelastic light scatter (Raman signal) from the defined ROI. In this method if excitation was blocked there would be no input anymore to analyse image or signal. Therefore the detection is blocked. Raman signal is collected and averaged over all blood pixels in the defined ROI by distribution of Raman excitation laser power over the defined ROI area instead directing it to a fixed point.

Using 1 laser: the Raman excitation beam is zoomed to the defined ROI. Part of the excitation beam is used to generate elastic light scatter for image analysis of the defined ROI and to detect skin and blood; another part is used for inelastic light scatter (Raman signal) from the defined ROI. Filtering (S3) is used for low frequency region and high frequency region. From the high frequency region a WPR is determined and monitored (S5) using filtering (S4). Therefrom skin or blood pixels can be detected (S6) to trigger the detection. Raman signal is collected and averaged over all blood pixels in the ROI by distribution of Raman excitation laser power over the defined ROI area instead of directing it to a fixed point.

The WPR determination can be done by read out of corresponding CCD pixels or spectral filtering (S3). Further, from the low frequency region (the so-called fingerprint) a PLS analysis can be made (S7) which allows the determination of the blood content in the defined ROI (S8).

FIG. 5 diagrammatically shows an embodiment of the analysis apparatus according to the invention including an optical separation system. A laser at λ₁ forms the radiation source that is used for confocal imaging and simultaneously for Raman excitation. The beam is split in two by the optical separation system (sep) formed by an (e.g. 20-80%) beam splitter (BS1). Part is used for confocal imaging, the other part is used for Raman excitation. The monitoring beam (irb) is linearly polarised by the polarising beam splitter (PBS). The scanning beam path in the confocal video microscope is deflected in x-y plane by the Θ-Φ mirror to form the image. Lenses L1 and L2 are used for beam expansion and L2 is used to image the central part of the Θ-Φ mirror on to the entrance pupil of the microscope objective (mo). In this way laser light reflected of the Θ-Φ mirror always enters the objective at the same position, irrespective of the actual Θ-Φ position of the Θ-Φ mirror.

The linearly polarised monitoring beam (λ₁; irb) is transformed to circularly polarised light by the quarter wave plate λ/4. The Raman excitation beam is reflected at the high pass filter (HPF) and directed towards the objective via the mirrors (M1, M2) and reflecting beamsplitter (BS2). On the return path reflected light from the object is transformed to linearly polarised light again however, shifted by 90° orientation, with respect to the polarisation orientation of the incoming beam. The transmitted light (partly the monitoring beam and partly the elastically scattered Raman light) trough the reflecting beam splitter (BS2) is then deflected by the polarising beam splitter (PBS) towards the APD detector to form the image and the Raman spot in the image. Elastically and inelastically scattered Raman light from the object is reflected at the BS2. The inelastically scattered Raman light (λ_{R}) is transmitted through the high pass filter HPF and directed towards the Raman detection path. The beamsplitter (BS2) can be exchanged by a spot reflector.

As described above regarding the first embodiment shown in FIG. 1 a control unit (ctrl) is provided for control of the excitation system (exs) and/or the detection system (dsy) based on information received from the imaging system (opd) in the way described above.

FIG. 6 diagrammatically shows a further embodiment of the analysis apparatus according to the invention wherein the monitoring system is an orthogonal polarised spectral imaging arrangement. This embodiment combines imaging by OPSI and Raman spectroscopy. For orthogonal polarised spectral imaging (OPSI) a light source (ls) is used at a specific wavelength band. To achieve this a white light source is filtered by a band pass filter (λ-Ftr). The light is linearly polarised by the polariser (P) and is then focused in the object by the objective lens (Obj). The reflected light is detected through an analyser at orthogonal polarisation orientation. This means that only depolarised light is detected which originates from multiply (diffusely) scattered light deep in the turbid object (tissue). The back scattering of these photons produces a sort of ‘backlight illumination’ which gives a more or less homogenous brightness in the image at the CCD detector (CCD). By proper selection of the wavelength (λ-Ftr) corresponding to (partly) absorption in shallow objects (such as capillaries in skin) these objects in contrast appear dark (through absorption) on a bright background. A Raman excitation beam can be coupled in the OPSI image in a similar fashion as in confocal imaging using a filter or other beam combination unit. The advantage of OPSI is especially its compactness and low cost.

Again, as described above regarding the first embodiment shown in FIG. 1 a control unit (ctrl) is provided for control of the excitation system (exs), which is in this embodiment separate from the light source (ls) for generating the monitoring beam (irb), based on information received from the imaging system (opd) in the way described above, and/or for control of the detection system (dsy).

The present invention allows the finding of a blood vessel in the image and the recording of Raman spectra of the blood vessel with a high SNR. Possible application areas of the invention are local analysis of a composition, such as for chip remote analysis of materials, non-invasive blood analysis or fast online analysis processes in production environments.

1. An analysis apparatus, in particular a spectroscopic analysis apparatus, for analysing an object comprising:
an excitation system for emitting an excitation beam to excite a target region,
a beam separation unit for separating at least part of elastically scattered radiation from inelastically scattered radiation, said scattered radiation being generated by the excitation beam at the target region,
a monitoring system for generating an image of the target region using the elastically scattered or the inelastically scattered radiation and for defining a region of interest in said image,
a detection system for detecting scattered radiation from the defined region of interest generated by the excitation beam and
a control unit for controlling the excitation system such that the defined region of interest of the target region is excited and/or for controlling the detection system such that only signals from the defined region of interest are detected.

2. An analysis apparatus as claimed in claim 1,
wherein said monitoring system is adapted to distinguish between different image portions using contrast information in the image.

3. An analysis apparatus as claimed in claim 1,
wherein said monitoring system is adapted to distinguish between different image portions using spectral information in the detected scattered radiation.

4. An analysis apparatus as claimed in claim 1,
wherein said detection system comprises a filter for separating high frequency spectral portions in a Raman signal, in particular portions comprising contributions from protein and water, from low frequency spectral portions, in particular a fingerprint spectral region.

5. An analysis apparatus as claimed in claim 1,
wherein said monitoring system is adapted for emitting a monitoring beam to image the target region.

6. An analysis apparatus as claimed in claim 5,
further comprising a radiation source to emit an output beam and an optical separation system to separate the monitoring beam and the excitation beam from the output beam.

7. An analysis apparatus as claimed in claim 1,
wherein said monitoring system includes a confocal scanning laser microscope and said detection system has a confocal relationship with the confocal scanning laser microscope.

8. An analysis apparatus as claimed in claim 1,
wherein said monitoring system includes an orthogonal polarised spectral imaging arrangement.

9. An analysis apparatus as claimed in claim 1,
wherein said control system is adapted for controlling said excitation system to distribute the laser power over the defined region of interest

10. An analysis apparatus as claimed in claim 1,
wherein said control system is adapted for controlling said detection system to block unwanted signals from parts of the defined region of interest and to detect only wanted signals from the defined region of interest.

11. An analysis method, in particular a spectroscopic analysis method, for analysing an object comprising the steps of:
emitting an excitation beam to excite a target region,
separating at least part of elastically scattered radiation from inelastically scattered radiation, said scattered radiation being generated by the excitation beam at the target region,
generating an image of the target region using the elastically scattered or the inelastically scattered radiation,
defining a region of interest in said image,—controlling the excitation system such that the defined region of interest of the target region is excited and/or controlling the detection system such that signals from the defined region of interest of the target region are detected, and
detecting scattered radiation from the defined region of interest generated by the excitation beam.
